(19) United States
(12) Patent Application Publication
(10) Pub. No.: US 2022/0369929 A1
(43) Pub. Date: Nov. 24, 2022

(54) SYSTEM AND METHOD FOR MEASURING BIO INFORMATION

(71) Applicant: SB Solutions Inc., Ulju-gun (KR)

(72) Inventors: Seungup Seo, Ulju-gun (KR); Namhwan Sung, Ulju-gun (KR)

(73) Assignee: SB Solutions Inc., Ulju-gun (KR)

(21) Appl. No.: 17/399,873

(22) Filed: Aug. 11, 2021

(30) Foreign Application Priority Data
May 18, 2021 (KR) 10-2021-0063883

(51) Int. Cl. A61B 5/00; A61B 5/11

(52) U.S. Cl. CPC A61B 5/0031; A61B 5/0008; A61B 5/746; A61B 5/742; A61B 5/11; A61B 5/6847; A61B 2562/0271; A61B 2560/0223

(57) ABSTRACT

Disclosed are a system and method for measuring bio information. The system for measuring bio information may include an external device configured to transmit wireless power from an outside of a body to an inside of the body, an implant device inserted into the body and configured to drive a sensing circuit by using the wireless power transmitted by the external device, measure bio data within the body by using the driven sensing circuit, calculate bio information based on the measured bio data, and transmit the calculated bio information to a smart device or the external device outside the body, and the smart device configured to transmit the bio information to a cloud server or display the bio information or output a warning alarm based on the bio information, when receiving the bio information from the implant device.

### CROSS-REFERENCES TO RELATED APPLICATIONS

This application is based on and claims priority under 35 U.S.C. 119 to Korean Patent Application No. 10-2021-0063883, filed on May 18, 2021, in the Korean intellectual property office, the disclosures of which are herein incorporated by reference in their entireties.

### TECHNICAL FIELD

The following description relates to a system and method for measuring bio information.

### BACKGROUND OF THE INVENTION

Examples in which adult-onset diseases, such as diabetes, hyperlipidemia and thrombosis, are increased are continuously reported. Such diseases need to be periodically measured using various bio sensors because it is important to continuously monitor and manage the diseases. A common type of bio sensor is a method of injecting, into a test strip, blood drawn from a finger and then quantizing an output signal by using an electrochemical method or a photometry method. Such an approach method causes a user a lot of pain because blood needs to be drawn every time.

For example, in order to manage diabetes of hundreds of millions of people around the globe, the most basic thing is to measure blood glucose. Accordingly, a blood glucose measurement device is an important diagnostic unit essential for a diabetes patient. Various blood glucose measurement devices are recently developed, but the most common method is a method of gathering blood by pricking a patient's finger and directly measuring a concentration of glucose within the blood. An invasive method includes a method of penetrating an invasive sensor into the skin, measuring a concentration of glucose through the invasive sensor for a given time, and measuring blood glucose by recognizing the blood glucose through an external reader.

In contrast, a non-invasive method includes a method using a light-emitting diode (LED)-photo diode (PD). However, the non-invasive method has low accuracy due to environmental elements and foreign substances, such as sweat or a temperature, because the LED-PD is attached to the skin.

The aforementioned information is to merely help understanding, and may include contents which do not form a part of a conventional technology and may not include contents which may be presented to those skilled in the art through a conventional technology.
[Prior Art Document Number]
Korean Patent No. 10-2185556

### SUMMARY

This summary is provided to introduce a selection of concepts in a simplified form that are further described below in the Detailed Description. This summary is not intended to identify key characteristics of the claimed subject matter, nor is it intended to be used as an aid in determining the scope of the claimed subject matter.

The present disclosure provides a system and method for measuring bio information, which have various structures using an implant device inserted into the body and an external device outside the body and/or a smart device.

There is provided a system for measuring bio information, including an external device configured to transmit wireless power from an outside of a body to an inside of the body, an implant device inserted into the body and configured to drive a sensing circuit by using the wireless power transmitted by the external device, measure bio data within the body by using the driven sensing circuit, calculate bio information based on the measured bio data, and transmit the calculated bio information to a smart device or the external device outside the body, and the smart device configured to transmit the bio information to a cloud server or display the bio information or output a warning alarm based on the bio information, when receiving the bio information from the implant device.

According to an aspect, the external device may include an external sensor configured to measure bio data within the body outside the body and a transmission module configured to transmit, to the cloud server, the bio data measured by the external sensor or bio information calculated based on the measured bio data.

According to another aspect, the cloud server may be configured to receive first bio information measured by the implant device through the smart device, receive the bio data measured by the external sensor through the external device, calculate second bio information based on the bio data received through the external device, and calibrate (or correct) the first bio information based on the second bio information.

According to yet another aspect, the smart device may be configured to receive the calibrated (or corrected) first bio information from the cloud server and to display the calibrated (or corrected) first bio information or output a warning alarm based on the calibrated (or corrected) first bio information.

There is provided a system for measuring bio information, including an implant device inserted into a body and configured to drive a sensing circuit by using wireless power transmitted by an external device, measure first bio data within the body by using the driven sensing circuit, calculate first bio information based on the measured first bio data, and transmit the calculated first bio information to the external device, and the external device configured to transmit the wireless power from an outside of the body to the implant device inserted into the body, measure, outside the body, second bio data within the body through the external sensor, receive the first bio information from the implant device, and transmit the first bio information and the second bio data to a cloud server.

According to an aspect, the system for measuring bio information may further include a cloud server configured to receive the first bio information and the second bio data from the external device, calculate second bio information based on the second bio data, calibrate (or correct) the first bio information based on the second bio information, and transmit the calibrated (or corrected) first bio information to the external device.

According to another aspect, the external device may be configured to receive the calibrated (or corrected) first bio information transmitted by the cloud server and to output a warning alarm based on the calibrated (or corrected) first bio information.

There is provided a system for measuring bio information, including an implant device inserted into a body and configured to drive a sensing circuit by using wireless power transmitted by a smart device, measure bio data within the body by using the driven sensing circuit, calculate bio information based on the measured bio data, and transmit the calculated bio information to the smart device, and the smart device configured to receive the bio information from the implant device and transmit the bio information to a cloud server, display the bio information or output a warning alarm based on the bio information.

According to an aspect, the sensing circuit may include both a signal source and a detector as sensing circuits having an oscillator type.

According to another aspect, the implant device may operate under the control of Bluetooth low energy (BLE) or included in the implant device or a micro controller unit (MCU) included in ultra low power (ULP) WiFi for communication toward the outside of the body.

According to yet another aspect, the implant device may include a temperature sensor configured to measure a temperature within the body, and the implant device may calculate bio information further based on an output value of the temperature sensor.

According to yet another aspect, the implant device may be configured to receive activity information of an object outside the body, and to calculate bio information further based on the received activity information.

There is provided a method of measuring, by an implant device inserted into a body, bio information, including receiving wireless power from an external device outside the body; driving a sensing circuit by using the wireless power; measuring bio data within the body by using the driven sensing circuit; calculating bio information based on the measured bio data; and transmitting the bio information to a smart device or the external device.

There is provided a method of measuring, by an implant device inserted into a body, bio information, including receiving wireless power from a smart device outside the body; driving a sensing circuit by using the wireless power; measuring bio data within the body by using the driven sensing circuit; calculating bio information based on the measured bio data; and transmitting the bio information to the smart device, wherein the smart device transmits the bio information to a cloud server.

There is provided a method of measuring, by an external device, bio information, including transmitting wireless power to an implant device inserted into the body; receiving first sensing information calculated by the implant device by using the wireless power; measuring, outside the body, bio data within the body through an external sensor included in the external device; transmitting the first sensing information and the bio data to a cloud server; receiving, from the cloud server, first sensing information calibrated (or corrected) based on the bio data; and outputting a warning alarm based on the calibrated (or corrected) first sensing information.

The system and method for measuring bio information, which have various structures using the implant device inserted into the body and the external device outside the body and/or the smart device can be provided.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a diagram illustrating an example of a system for measuring bio information according to an embodiment of the present disclosure.

FIGS. 2 to 4 are diagrams illustrating examples of a process of measuring bio information for each case in an embodiment of the present disclosure.

FIGS. 5 to 7 are diagrams illustrating examples of internal components of an implant device according to an embodiment of the present disclosure.

FIG. 8 is a diagram illustrating an example of internal components of an external device according to an embodiment of the present disclosure.

FIG. 9 is a block diagram illustrating an example of a computer device according to an embodiment of the present disclosure.

FIG. 10 is a flowchart illustrating an example of a method of measuring bio information according to Case 1 in an embodiment of the present disclosure.

FIG. 11 is a flowchart illustrating a first example of a method of measuring bio information according to Case 2 in an embodiment of the present disclosure.

FIG. 12 is a flowchart illustrating a second example of a method of measuring bio information according to Case 2 in an embodiment of the present disclosure.

FIG. 13 is a flowchart illustrating a method of measuring bio information according to Case 3 in an embodiment of the present disclosure.

### DETAILED DESCRIPTION

While illustrative embodiments have been illustrated and described, it will be appreciated that various changes can be made therein without departing from the spirit and scope of the invention.

Hereinafter, embodiments are described in detail with reference to the accompanying drawings. However, the embodiments may be changed in various ways, and the scope of right of this patent application is not limited or restricted by such embodiments. It is to be understood that all changes, equivalents and substitutions of the embodiments are included in the scope of right.

Terms used in embodiments are merely used for a description purpose and should not be interpreted as intending to restrict the present disclosure. An expression of the singular number includes an expression of the plural number unless clearly defined otherwise in the context. In this specification, it should be understood that a term, such as “include” or “have”, is intended to designate the presence of a characteristic, a number, a step, an operation, a component, a part or a combination of them described in the specification, and does not exclude the existence or possible addition of one or more other characteristics, numbers, steps, operations, components, parts, or combinations of them in advance.

All terms used herein, including technical or scientific terms, have the same meanings as those commonly understood by a person having ordinary knowledge in the art to which an embodiment pertains, unless defined otherwise in the specification. Terms, such as those commonly used and defined in dictionaries, should be construed as having the same meanings as those in the context of a related technology, and are not construed as being ideal or excessive unless explicitly defined otherwise in the specification.

Furthermore, in describing the present disclosure with reference to the accompanying drawings, the same component is assigned the same reference numeral regardless of its reference numeral, and a redundant description thereof is omitted. In describing an embodiment, a detailed description of a related known art will be omitted if it is deemed to make the gist of the embodiment unnecessarily vague.

Furthermore, in describing components of an embodiments, terms, such as a first, a second, A, B, (a), and (b), may be used. Such terms are used only to distinguish one component from the other component, and the essence, order, or sequence of a corresponding component is not limited by the terms. When it is said that one component is “connected”, “combined”, or “coupled” to the other component, the one component may be directly connected or coupled to the other component, but it should also be understood that a third component may be “connected”, “combined”, or “coupled” between the two components.

A component included in any one embodiment and a component including a common function are described using the same name in another embodiment. Unless described otherwise, a description written in any one embodiment may be applied to another embodiment, and a detailed description in a redundant range is omitted.

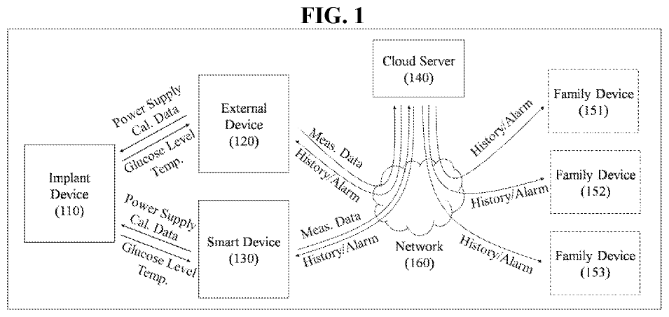

FIG. 1 is a diagram illustrating an example of a system for measuring bio information according to an embodiment of the present disclosure. The system for measuring bio information according to the present disclosure may include an implant device 110, an external device 120, a smart device 130, a cloud server 140 and a plurality of family devices 151 to 153. In this case, in some embodiments, only one of the external device 120 and the smart device 130 may be included. The external device 120 and/or the smart device 130 may communicate with the cloud server 140 over a network 160. Furthermore, in some embodiments, the plurality of family devices 151 to 153 may be omitted. FIG. 1 illustrates three family devices, such as the plurality of family devices 151 to 153, but the number of family device is not limited to three.

The implant device 110 may include a signal source for outputting a signal for measuring bio information and a detector for detecting a reflected and returned signal. The implant device 110 may be inserted into the body of an object. The object may be a person, but may include an animal other than a person. The implant device 110 may include a sensing circuit having an oscillator type. The signal source and the detector may be included in the sensing circuit. Furthermore, the implant device 110 may operate based on power wirelessly transmitted by the external device 120 or the smart device 130, and may receive and use calibration data (Cal. Data) from the external device 120 or the smart device 130. For example, the implant device 110 may output a signal through the signal source by using power wirelessly transmitted by the external device 120 or the smart device 130, and may detect a reflected and returned signal by using the detector. In this case, the implant device 110 may calculate bio information (e.g., a glucose level in FIG. 1) based on sensing data, that is, detected data, and received calibration data, and may transmit the calculated bio information to the external device 120 or the smart device 130.

The external device 120 and the smart device 130 may basically have the same role for wireless power transfer toward the implant device 110 and data collection from the implant device 110. Furthermore, the external device 120 or the smart device 130 may upload, onto the cloud server 140, data measured and collected by the implant device 110 over the network 160. The cloud server 140 may store and manage data uploaded for each user. For example, the cloud server 140 may transmit, to the external device 120 or the smart device 130, a history of uploaded data or an alarm based on uploaded data based on data uploaded for each user. To this end, the cloud server 140 may include a function for analyzing uploaded data.

The system for measuring bio information illustrated in FIG. 1 may be used in three cases as in Table 1.

TABLE 1

| | IMPLANT | EXTERNAL | SMART | | |
|---|---|---|---|---|---|
| CLASSIFICATION | DEVICE | DEVICE | DEVICE | NETWORK | OTHERS |
| Case 1 | Implant | Power | Wearable | Cloud | Family |
| | device | supply | device | | device |
| | | | Smartphone | | |
| Case 2 | Implant | Power | — | | |
| | device | supply + | Wearable | | |
| | | External | device | | |
| | | sensor | Smartphone | | |
| Case 3 | Implant | — | Smartphone | | |
| | device | | | | |
| Case 4 | | External | | | |
| | | sensor | | | |

In Cases 1 and 3, the implant device 110 may play a role to calculate and provide bio information. In Case 2, both the implant device 110 and the cloud server 140 may calculate bio information. In this case, in Case 2, the implant device 110 may autonomously calculate and provide first bio information. The cloud server 140 may receive information from an external sensor which may be included in the external device 120, and may calculate second bio information. The first bio information calculated by the implant device 110 and the second bio information calculated by the cloud server 140 may be compared and used to determine whether comparison data (i.e., a difference between the first bio information and the second bio information) is within a margin of error. When the comparison data is not within the margin of error, bio information may be measured and calculated again. Furthermore, in Case 4, the external device 120 including the external sensor may measure bio data, and may transmit, to the cloud server 140, the measured bio data or bio information calculated based on the measured bio data. Case 4 may mean a case where the implant device 110 and the smart device 130 are not present.

Table 1 illustrates an example in which the smart device 130 may be implemented in a form, such as a wearable device or a smartphone, but the present disclosure is not limited thereto.

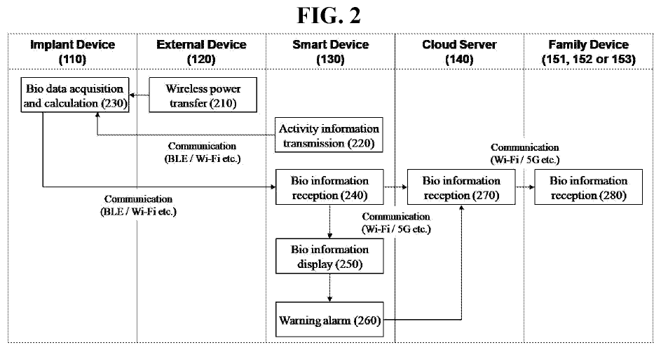

FIGS. 2 to 4 are diagrams illustrating examples of a process of measuring bio information for each case in an embodiment of the present disclosure.

In the present embodiments, sensing data obtained by the implant device 110 by detecting the sensing data through the sensing circuit and sensing data obtained by the external device 120 through the external sensor are described as “bio data.” Data processed in a form which may be provided to a user, such as a level of a target material (e.g., glucose) calculated by the implant device 110 and/or the cloud server 140 further based on activity information of the user, calibration data, etc., is described as “bio information.”

Communication between the external device 120 and/or the smart device 130 and the implant device 110 may be performed through Bluetooth low energy (BLE), WiFi, etc., but the present disclosure is not limited thereto. Furthermore, communication between the external device 120 and/or the smart device 130 and the cloud server 140 and/or communication between the cloud server 140 and the family devices 151 to 153 may be performed through WiFi or a 5-th generation mobile communication technology (5G), but the present disclosure is not limited thereto.

Case 1 described with reference to FIG. 2 illustrates an example of a case where both the external device 120 and the smart device 130 are present and the implant device 110 performs communication in order to transfer bio information to the smart device 130. In this case, the bio information (e.g., a glucose level) may be calculated by the implant device 110. The external device 120 may be implemented to have a function for transmitting wireless power to the implant device 110.

Wireless power transfer 210 may be an example of a process of wirelessly transmitting, by the external device 120, power to the implant device 110. The wireless power transfer (WPT) technology has already been well known, and thus a detailed description thereof is omitted.

Activity information transmission 220 may be an example of a process of transmitting, by the smart device 130, measured activity information of a user to the implant device 110.

Bio data acquisition and calculation 230 may be an example of a process of obtaining, by the implant device 110, bio data by using power wirelessly transmitted by the external device 120 and calculating bio information by using the obtained bio data and the activity information received from the smart device 130.

Bio information reception 240 may be an example of a process of receiving, by the smart device 130, the bio information from the implant device 110.

Bio information display 250 may be an example of a process of outputting, by the smart device 130, the received bio information through an output device included in the smart device 240. In general, the output of the bio information may be performed as a visual output through a display, but does not exclude an auditory output through a speaker.

Warning alarm 260 may be an example of a process of outputting, by the smart device 130, a warning alarm in a specific situation. In this case, the specific situation may include a situation in which a level of bio information is less than a first threshold value or is greater than a second threshold value. Furthermore, the warning alarm may also be transmitted to the cloud server 140 and included in a history of bio information for a user.

Bio information reception 270 may be an example of a process of receiving, by the cloud server 140, bio information from the smart device 130. In this case, the cloud server 140 may generate and/or update a history of bio information for a specific user by storing the received bio information in association with an identifier of the smart device 130 and/or an identifier of the user of the smart device 130. In this case, the identifier of the implant device 110 and/or the identifier of the external device 120 may be stored in the cloud server 140 in further association with the bio information.

Bio information reception 280 may be an example of a process of receiving, by the family device 151, 152 or 153, the information through the cloud server 140. The family device 151, 152 or 153 may be a device different from the smart device 130 of a user or may be a device of the person concerned (e.g., a hospital or doctor associated with the user).

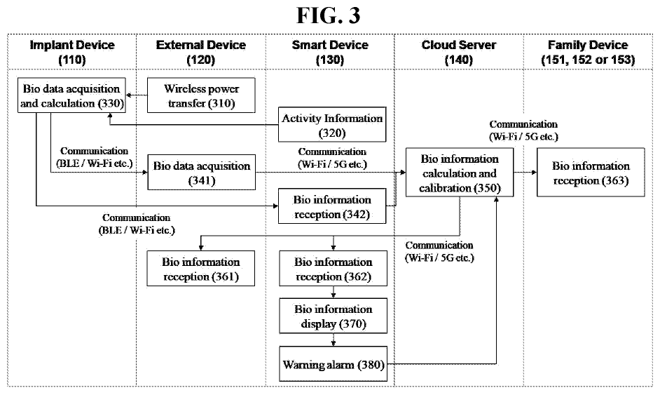

In Case 2 described with reference to FIG. 3, the external device 120 may further include an external sensor.

Wireless power transfer 310 may be an example of a process of wirelessly transmitting, by the external device 120, power to the implant device 110.

Activity information transmission 320 may be an example of a process of transmitting, by the smart device 130, measured activity information of a user to the implant device 110. If the smart device 130 is not present, the process of the activity information transmission 320 may be omitted.

Bio data acquisition and calculation 330 may be an example of a process of obtaining, by the implant device 110, bio data by using power wirelessly transmitted by the external device 120 and calculating first bio information based on the obtained bio data and the activity information received from the smart device 130. If the smart device 130 is not present, the activity information may not be used. In another embodiment, the activity information may be obtained through a sensor (e.g., a gyro sensor) which may be included in the external device 120 and transmitted to the implant device 110.

Bio data acquisition 341 may be an example of a process of obtaining, by the external device 120, bio data by using the external sensor further included in the external device 120. The implant device 110 may scan electromagnetic waves to the surroundings of the sensor in a dense frequency over a wide band under the skin, and may precisely measure a change in permittivity attributable to a change in a target material through the analysis of characteristics of an electromagnetic (EM) reflected for each frequency. The external sensor may be an electromagnetic (EM)-based sensor attached to a surface of the skin outside the body, and may measure bio data, such as blood glucose, in a non-invasive manner by analyzing a change in electromagnetic waves that penetrate an interstitial fluid layer based on a change in the coupling of the electromagnetic waves that penetrate the body by using a plurality of EM-based sensors in a surface of the skin outside the body.

Bio information reception 342 may be an example of a process of receiving, by the smart device 130, the first bio information from the implant device 110. If the smart device 130 is not present, the process of the bio information reception 342 may be omitted.

Bio information calculation and calibration 350 may be an example of a process of calculating, by the cloud server 140, second bio information based on the bio data received from the external device 120 and calibrating (or correcting) the first bio information received from the smart device 130 based on the second bio information.

In the sub-case in which the smart device 130 is not present, the process of the bio data acquisition 341 by the external device 120 may include a process of receiving the first bio information from the implant device 120.

In this case, bio information reception 361 may be a process of receiving, by the external device 120, the second bio information generated by the cloud server 140 based on the bio data received from the external device 120. In this case, the external device 120 may check whether comparison data is included in a margin of error by comparing the first bio information and the second bio information. When the comparison data is not included in the margin of error, the external device 120 may restart the process of the wireless power transfer 310 in order to calculate the first bio information and the second bio information again.

Bio information reception 362 may be an example of a process of receiving, by the smart device 130, bio information from the cloud server 140. In this case, the received bio information may be the first bio information calibrated (or corrected) in the process of the bio information calculation and calibration 350.

Bio information reception 363 may be an example of a process of receiving, by the family device 151, 152 or 153, the bio information from the cloud server 140.

A process of bio information display 370 and a process of warning alarm 380 may correspond to the process of the bio information display 250 and the process of the warning alarm 260 described with reference to FIG. 2.

In a sub-case in which the smart device 130 is not present, the process of the bio information display 370 and the process of the warning alarm 380 may be omitted. In this case, the cloud server 140 may calculate bio information based on received bio data, and may transmit a warning alarm to the external device 120 in a specific situation. In this case, the external device 120 may notify a user of the received warning alarm by outputting the received warning alarm. As described above, the specific situation may include a situation in which a level of bio information is less than a first threshold value or is greater than a second threshold value.

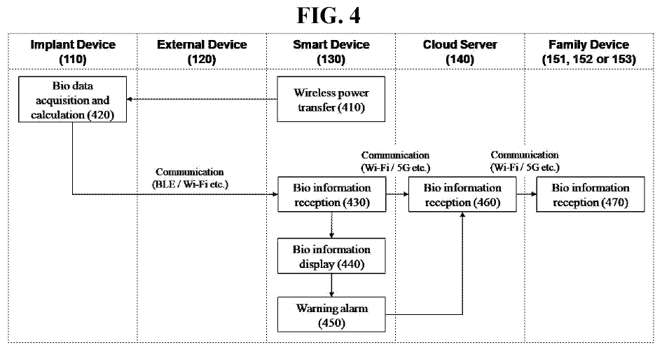

Case 3 described with reference to FIG. 4 illustrates an example of a case where the external device 120 is not present and the implant device 110 performs communication in order to transfer bio information to the smart device 130.

Wireless power transfer 410 may be an example of a process of wirelessly transmitting, by the smart device 130, power to the implant device 110.

Bio data acquisition and calculation 420 may be an example of a process of obtaining, by the implant device 110, bio data by using power wirelessly transmitted by the smart device 130 and calculating bio information based on the obtained bio data.

Bio information reception 430 may be an example of a process of receiving, by the smart device 130, the bio information from the implant device 110.

A process of bio information display 440 and a process of warning alarm 450 may correspond to the process of the bio information display 250 and the process of the warning alarm 260 described with reference to FIG. 2.

Bio information reception 460 may be an example of a process of receiving, by the cloud server 140, the bio information from the smart device 130. The cloud server 140 may generate and/or update a history of bio information for a specific user by storing the received bio information in association with an identifier of the smart device 130 and/or an identifier of a user of the smart device 130. In this case, the identifier of the implant device 110 may be stored in the cloud server 140 in further association with the bio information.

Bio information reception 470 may be an example of a process of receiving, by the family device 151, 152 or 153, the bio information through the cloud server 140. As described above, the family device 151, 152 or 153 may be a device different from the smart device 130 of a user or may be a device of the person concerned (e.g., a hospital or doctor associated with the user).

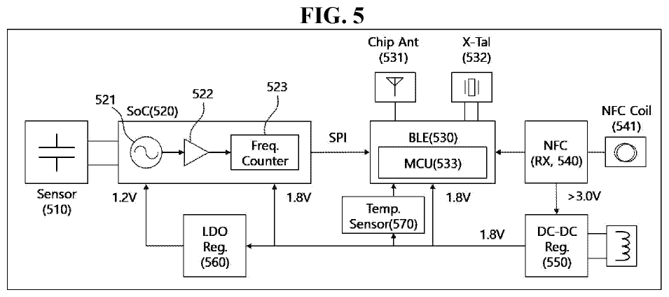

FIGS. 5 to 7 are diagrams illustrating examples of internal components of an implant device according to an embodiment of the present disclosure.

An implant device 110 according to an embodiment of FIG. 5 may include a sensor 510, a system on chip (SoC) 520, BLE 530, near field communication (NFC) (RX) 540, a DC-DC regulator 550, a low drop out (LDO) regulator 560 and a temperature sensor 570.

The SoC 520 may include an oscillator 521, an amplifier 522 and a frequency counter 523. The oscillator 521 may be used to generate a signal having an accurate frequency. The generated signal having the frequency may be output in order to measure a change in permittivity attributable to a change in a surrounding target material. The sensor 510 may detect a reflected signal. The amplifier 522 may transmit the detected signal to the frequency counter 523 by amplifying the detected signal. The frequency counter 523 is a circuit for calculating a frequency of a signal received from the amplifier 522, and may be a circuit for detecting a zero cross for an input signal.

Detected frequency data may be transmitted from the SoC 520 to a micro controller unit (MCU) 533 included in the BLE 530 through a serial peripheral interface (SPI), and may be transmitted to the external device 120 or the smart device 130 through an antenna (2.4 GHz chip antenna) 531 and/or an X-tal 532 of 32 MHz, which is connected to the BLE 530.

The external device 120 or the smart device 130 may transmit power for driving the implant device 120 through wireless power transfer. The NFC 540 included in the implant device 120 may receive power using an NFC coil 541. At this time, the NFC 540 may transfer power having a first voltage (e.g., a voltage between 3.0 V and 5.5 V) to the DC-DC regulator 550. The DC-DC regulator 550 may convert the power having the first voltage, received from the NFC 540, into power having a second voltage (e.g., 1.8 V) for an interface part (i.e., a part for an SPI) of the MCU 533 of the BLE 530 and the SoC 520. The embodiment of FIG. 5 illustrates an example in which power of 1.8 V is transmitted to the BLE 530 and the SoC 520. Furthermore, the LDO regulator 560 may convert the power having the second voltage, generated and transmitted by the DC-DC regulator 550, into power having a third voltage (e.g., 1.2 V) for the cores (i.e., the oscillator 521, the amplifier 522 and the frequency counter 523) of the SoC 520. The embodiment of FIG. 5 illustrates an example in which the power of 1.2 V generated by the LDO regulator 560 is transmitted to the SoC 520.

Furthermore, the power having the second voltage may also be transmitted to the temperature sensor 570. A temperature value measured by the temperature sensor 580 may also be transmitted to the external device 120 or the smart device 130 through the BLE 530.

The implant device 110 may be controlled by the MCU 533 included in the BLE 530.

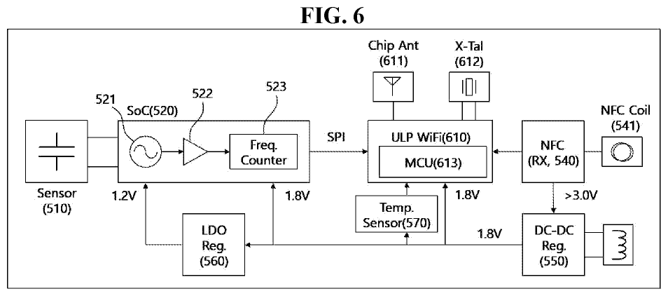

The implant device 110 according to an embodiment of FIG. 6 may include ultra low power (ULP) WiFi 610 instead of the BLE 530 described in the embodiment of FIG. 5. In this case, an MCU 613 included in the ULP WiFi 610 instead of the MCU 533 included in the BLE 530 may perform the same function. As in FIG. 5, the ULP WiFi 610 may be connected to an antenna (i.e., a 2.4 GHz chip antenna 611 and/or an X-tal 612 of 32 MHz and 40 MHz) for communicating with the external device 120 and/or the smart device 130. For example, the MCU 613 may transmit, to the external device 120 or the smart device 130, frequency data received from the SoC 520 and a temperature value received from the temperature sensor 570 through the antenna.

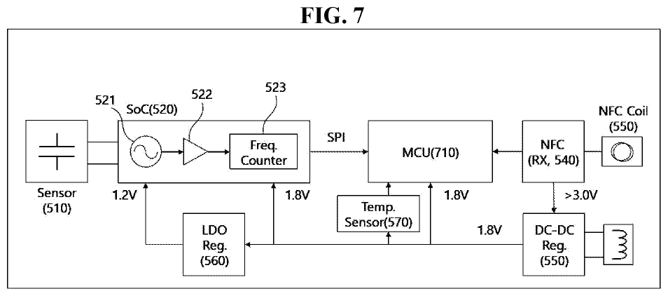

The implant device 110 according to an embodiment of FIG. 7 may include an MCU 710 instead of the BLE 530 or the ULP WiFi 610 described in the embodiment of FIG. 5. In this case, the MCU 710 may transmit, to the external device 120 or the smart device 130, frequency data received from the SoC 520 and a temperature value received from the temperature sensor 570 through the NFC 540.

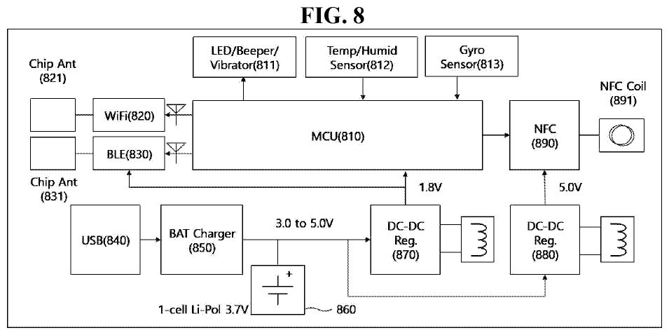

FIG. 8 is a diagram illustrating an example of internal components of an external device according to an embodiment of the present disclosure. The external device 120 may include an MCU 810, WiFi 820, BLE 830, a universal serial bus (USB) 840, a battery (BAT) charger 850, a battery 860, a first DC-DC regulator 870, a second DC-DC regulator 880 and NFC (TX) 890.

The external device 120 may operate under the control of the MCU 810. The WiFi 820 may be a WiFi module. The external device 120 may communicate with the client server 140 by using the WiFi 820 under the control of the MCU 810. Likewise, the external device 120 may communicate with the implant device 110 by using the BLE 830 under the control of the MCU 810. To this end, the WiFi 820 and the BLE 830 may be connected to respective antennas (e.g., 2.4 GHz chip antennas 821 and 831). The WiFi 820 and the BLE 830 are merely examples, and the present disclosure is not limited thereto. For example, as described above, the WiFi 820 may be used to communicate with the implant device 110. A 5-th generation mobile communication technology may be used for communication with the client server 140. In some embodiments, the WiFi 820 and the BLE 830 may be used for the external device 120 to communicate with the smart device 130.

The battery 860 may be charged through the USB 840 and the battery charger 850. For example, a 1-cell 3.7V lithium polymer battery may be used as the battery 860, but the present disclosure is not limited thereto. Power having a fourth voltage (e.g., a voltage between 3.0 V and 5.0 V) may be transmitted to the first DC-DC regulator 870 and the second DC-DC regulator 880 through the battery charger 850 or the battery 860. The first DC-DC regulator 870 may convert the power having the fourth voltage into power having a fifth voltage (e.g., 1.8 V), and may transmit the power having the fifth voltage to the MCU 810, the WiFi 820, and the BLE 830. Furthermore, the second DC-DC regulator 880 may convert the power having the fourth voltage into power having a sixth voltage (e.g., 5.0 V), and may transmit the power having the sixth voltage to the NFC (TX) 890. The NFC 890 may transmit the power having the sixth voltage to the implant device 110 through an NFC coil 891.

The external device 120 may further include an output device 811, a temperature/humidity sensor 812 and/or a gyro sensor 813.

For example, the MCU 810 may be connected to the output device 811 for providing a user with visual, auditory and/or tactile information. The output device 811 may include a light-emitting diode (LED), a beeper and/or a vibrator as illustrated in FIG. 8, but the present disclosure is not limited thereto. The output device 811 may be used to provide a user with a warning alarm.

Furthermore, the MCU 810 may be connected to the temperature/humidity sensor 812. If the temperature sensor 570 included in the implant device 120 is used to measure a body temperature, the temperature/humidity sensor 812 may be used to measure information on a surrounding environment of a user. A temperature value and/or a humidity value measured by the temperature/humidity sensor 812 may be transmitted to the implant device 110, the smart device 130 and/or the cloud server 140 through the MCU 810, the WiFi 820 and the BLE 830.

Furthermore, the MCU 810 may be connected to the gyro sensor 813. The gyro sensor 813 may be used to generate activity information of a user based on an angular speed of the external device 120. An embodiment in which the smart device 130 generates activity information and transmits the activity information to the implant device 110 has been described above, but it may be easily understood that the external device 120 may provide activity information to the implant device 110 based on the gyro sensor 813.

The smart device 130, the cloud server 140, and each of the plurality of family devices 151 to 153 may be implemented as at least one computer device.

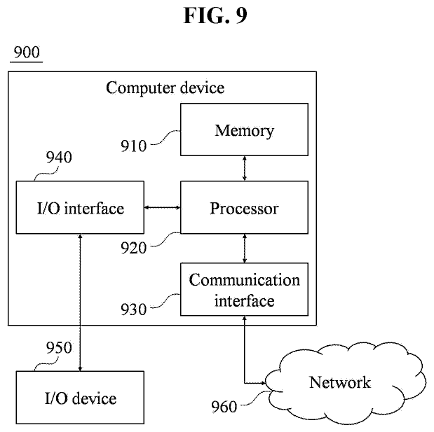

FIG. 9 is a block diagram illustrating an example of a computer device according to an embodiment of the present disclosure. As illustrated in FIG. 9, a computer device 900 may include a memory 910, a processor 920, a communication interface 930 and an input/output (I/O) interface 940.

The memory 910 is a computer-readable recording medium, and may include permanent mass storage devices, such as a random access memory (RAM), a read only memory (ROM) and a disk drive. In this case, the permanent mass storage device, such as the ROM or the disk drive, may be included in the computer device 900 as a separate permanent storage device different from the memory 910. Furthermore, an operating system and at least one program code may be stored in the memory 910. Such software components may be loaded onto the memory 910 from a computer-readable recording medium separate from the memory 910. Such a separate computer-readable recording medium may include computer-readable recording media, such as a floppy drive, a disk, a tape, a DVD/CD-ROM drive, and a memory card. In another embodiment, the software components may be loaded onto the memory 910 through the communication interface 930 not the computer-readable recording medium. For example, the software components may be loaded onto the memory 910 of the computer device 900 based on a computer program installed by filed received over a network 960. The network 960 may include network 160 described with reference to FIG. 1.

The processor 920 may be configured to process instructions of a computer program by performing basic, arithmetic, logic and I/O operations. The instruction may be provided to the processor 920 by the memory 910 or the communication interface 930. For example the processor 920 may be configured to execute instructions received based on a program code stored in a recording device, such as the memory 910.

The communication interface 930 may provide a function for enabling the computer device 900 to communicate with another device over the network 960. For example, a request, an instruction, data, a file, etc. generated by the processor 920 of the computer device 900 based on a program code stored in a recording device, such as the memory 910, may be transmitted to other devices over the network 960 under the control of the communication interface 930. Inversely, a signal, an instruction, data, a file, etc. from another device may be received by the computer device 900 through the communication interface 930 of the computer device 900 over the network 960. The signal, the instruction, the data, etc. received through the communication interface 930 may be transmitted to the processor 920 or the memory 910. The file, etc. received through the communication interface 930 may be stored in a storage medium (the aforementioned permanent storage device) which may be further included in the computer device 900.

The I/O interface 940 may be means for an interface with an I/O device 950. For example, the input device may include a device, such as a microphone, a keyboard or a mouse. Furthermore, the output device may include a device, such as a display or a speaker. For another example the I/O interface 940 may be means for an interface with a device in which functions for input and output are integrated into one, such as a touch screen. The I/O device 950 may be configured as a single device along with the computer device 900.

Furthermore, in other embodiments, the computer device 900 may include components smaller or greater than the components of FIG. 9. However, most of conventional components do not need to be clearly illustrated. For example, the computer device 900 may be implemented to include at least some of the I/O device 950 or may further include other components, such as a transceiver and a database.

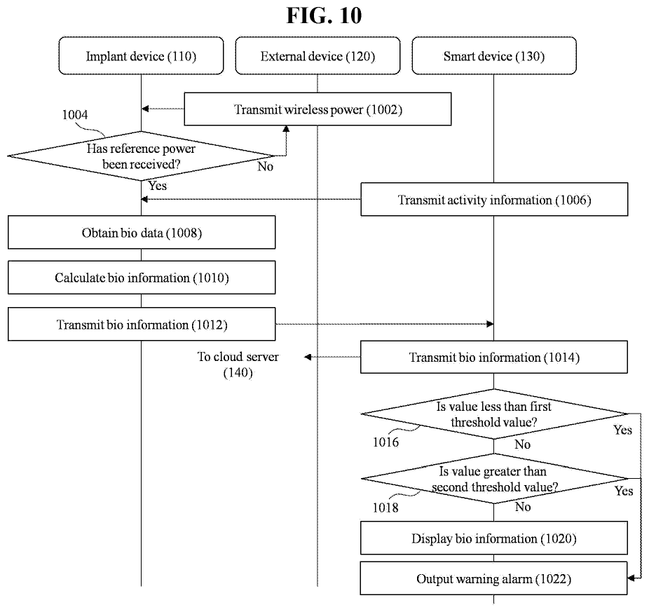

FIG. 10 is a flowchart illustrating an example of a method of measuring bio information according to Case 1 in an embodiment of the present disclosure. FIG. 10 illustrates the implant device 110, the external device 120 and the smart device 130.

In step 1002, the external device 120 may transmit wireless power to the implant device 110. In other words, the external device 120 may transmit wireless power from the outside of the body to the inside of the body.

In step 1004, the implant device 110 may determine whether reference power has been received. The external device 120 transmits the wireless power from the outside of the body to the inside of the body. At this time, if the external device 120 does not accurately transmit the wireless power to a location of the implant device 110, the implant device 110 may not receive the reference power. In this case, a location of the external device 120 may be readjusted, and wireless power may be transmitted again. To this end, if the implant device 110 does not receive the reference power, step 1002 may be performed again. If the implant device 110 receives the reference power, step 1006 may be performed.

In step 1006, the smart device 130 may transmit activity information to the implant device 110. The activity information may be information obtained by the smart device 130 through a sensor (e.g., an accelerator, a gyro sensor or a proximity sensor).

In step 1008, the implant device 110 may obtain bio data. In this case, the implant device 110 may drive the sensing circuit by using the wireless power received from the external device 120, and may measure the bio data within the body by using the driven sensing circuit. In some embodiments, the sequence of step 1006 and step 1008 may be changed.

In step 1010, the implant device 110 may calculate bio information. In this case, the implant device 110 may calculate the bio information based on the measured bio data. In some embodiments, the activity information received in step 1006 may be further used to calculate the bio information.

In step 1012, the implant device 110 may transmit the bio information to the smart device 130.

In step 1014, the smart device 130 may transmit the bio information to the cloud server 140. As described above, the cloud server 140 may generate and/or update a history of bio information for a specific user by storing the bio information in association with an identifier of the smart device 130 and/or an identifier of the user of the smart device 130. In this case, the identifier of the implant device 110 and/or the identifier of the external device 120 may be stored in the cloud server 140 in further association with the bio information. Furthermore, the cloud server 140 may transmit at least some of a generated and/or updated history to the smart device 130 or the plurality of family devices 151 to 153, if necessary. Accordingly, a user or the person concerned can access the history of bio information easily and conveniently.

In step 1016, the smart device 130 may determine whether a value (e.g., a concentration level of a target material) based on the bio information is less than a first threshold value. In this case, step 1018 may be performed when the corresponding value is not less than the first threshold value, and step 1022 may be performed when the corresponding value is less than the first threshold value.

In step 1018, the smart device 130 may determine whether the value based on the bio information is greater than a second threshold value. In this case, step 1020 may be performed when the corresponding value is not greater than the second threshold value, and step 1022 may be performed when the corresponding value is greater than the second threshold value.

In step 1020, the smart device 130 may display the bio information. For example, the smart device 130 may provide the bio information to a user by displaying the bio information on the display included in the smart device 130. In some embodiments, the smart device 130 may also provide the bio information to user in the form of auditory information.

In step 1022, the smart device 130 may output a warning alarm. The warning alarm may be output in a visual, auditory and/or tactile manner. If the value based on the bio information is the first threshold value and is less than a low reference level, the smart device 130 may output a warning alarm in order to give warning of a low concentration of a target material. Inversely, if the value based on the bio information is the second threshold value and is greater than a high reference level, the smart device 130 may output a warning alarm in order to give warning of a high concentration of a target material.

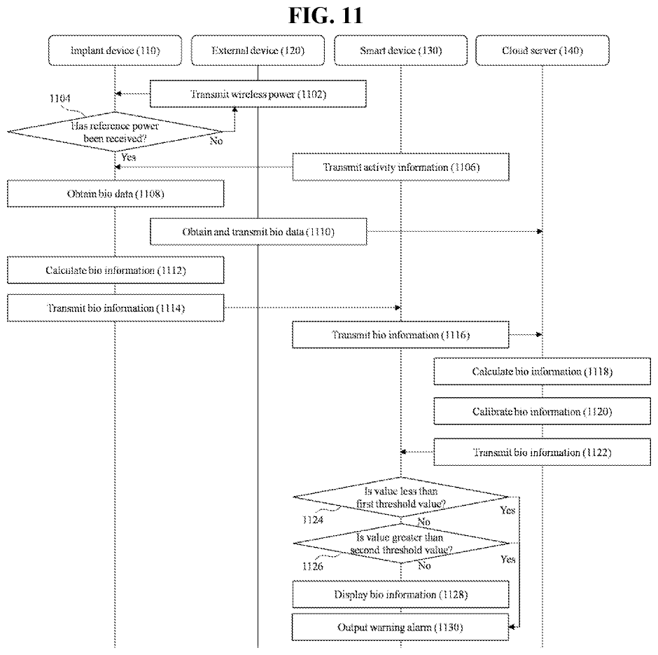

FIG. 11 is a flowchart illustrating a first example of a method of measuring bio information according to Case 2 in an embodiment of the present disclosure. FIG. 11 illustrates the implant device 110, the external device 120, the smart device 130 and the cloud server 140.

In step 1102, the external device 120 may transmit wireless power to the implant device 110. In other words, the external device 120 may transmit the wireless power from the outside of the body to the inside of the body.

In step 1104, the implant device 110 may determine whether reference power has been received. The external device 120 transmits the wireless power from the outside of the body to the inside of the body. At this time, if the external device 120 does not accurately transmit the wireless power to a location of the implant device 110, the implant device 110 may not receive the reference power. In this case, a location of the external device 120 may be readjusted, and the wireless power may be transmitted again. To this end, step 1102 may be performed again when the implant device 110 does not receive the reference power. Step 1106 may be performed when the implant device 110 receives the reference power.

In step 1106, the smart device 130 may transmit activity information to the implant device 110. The activity information may be information obtained by a sensor (e.g., an accelerator, a gyro sensor, or a proximity sensor) included in the smart device 130.

In step 1108, the implant device 110 may obtain bio data. In this case, the implant device 110 may drive the sensing circuit by using the wireless power received from the external device 120, and may measure the bio data within the body by using the driven sensing circuit. In some embodiments, the sequence of step 1006 and step 1008 may be changed.

In step 1110, the external device 120 may obtain and transmit bio data. In this case, the external device 120 may measure, outside the body, the bio data within the body through the external sensor, and may transmit the measured bio data to the cloud server 140. A process for the external device 120 to obtain and transmit the bio data may be performed separately from a process for the implant device 110 to obtain the bio data. In other words, step 1110 may be performed prior to step 1108 or may be performed after step 1112.

In step 1112, the implant device 110 may calculate bio information. In this case, the implant device 110 may calculate the bio information based on the measured bio data. In some embodiments, the activity information received in step 1006 may be further used to calculate the bio information.

In step 1114, the implant device 110 may transmit the bio information to the smart device 130.

In step 1116, the smart device 130 may transmit the bio information to the cloud server 140.

In step 1118, the cloud server 140 may calculate bio information. For example, the cloud server 140 may receive the bio data obtained and transmitted by the external device 120 in step 1110, and may calculate the bio information based on the received bio data.

In step 1120, the cloud server 140 may calibrate (or correct) the bio information. For example, the cloud server 140 may calibrate (or correct) the bio information, received from the smart device 130, based on the bio information calculated by the cloud server 140.

In step 1122, the cloud server 140 may transmit bio information to the smart device 130. In this case, the transmitted bio information may be bio information calibrated (or corrected) in step 1120. In some embodiments, the cloud server 140 may generate and/or update a history of bio information for a specific user by storing the bio information in association with an identifier of the smart device 130 and/or an identifier of the user of the smart device 130. In this case, the identifier of the implant device 110 and/or the identifier of the external device 120 may also be stored in the cloud server 140 in further association with the bio information. Furthermore, the cloud server 140 may transmit at least some of a generated and/or updated history to the smart device 130 or the plurality of family devices 151 to 153, if necessary. Accordingly, a user or the person concerned can access the history of bio information easily and conveniently.

In step 1124, the smart device 130 may determine whether a value based on the bio information (e.g., a concentration level of a target material) is less than a first threshold value. In this case, step 1126 may be performed when the corresponding value is not less than the first threshold value, and step 1130 may be performed when the corresponding value is less than the first threshold value.

In step 1126, the smart device 130 may determine whether the value based on the bio information is greater than a second threshold value. In this case, step 1128 may be performed when the corresponding value is not greater than the second threshold value, and step 1130 may be performed when the corresponding value is greater than the second threshold value.

In step 1128, the smart device 130 may display the bio information. For example, the smart device 130 may provide the bio information to a user by displaying the bio information on the display included in the smart device 130. In some embodiments, the smart device 130 may also provide the bio information to the user in the form of auditory information.

In step 1130, the smart device 130 may output a warning alarm. The warning alarm may be output in a visual, auditory and/or tactile manner. If the value based on the bio information is the first threshold value and is less than a low reference level, the smart device 130 may output a warning alarm in order to give warning of a low concentration of a target material. Inversely, if the value based on the bio information is the second threshold value and is greater than a high reference level, the smart device 130 may output a warning alarm in order to give warning of a high concentration of a target material.

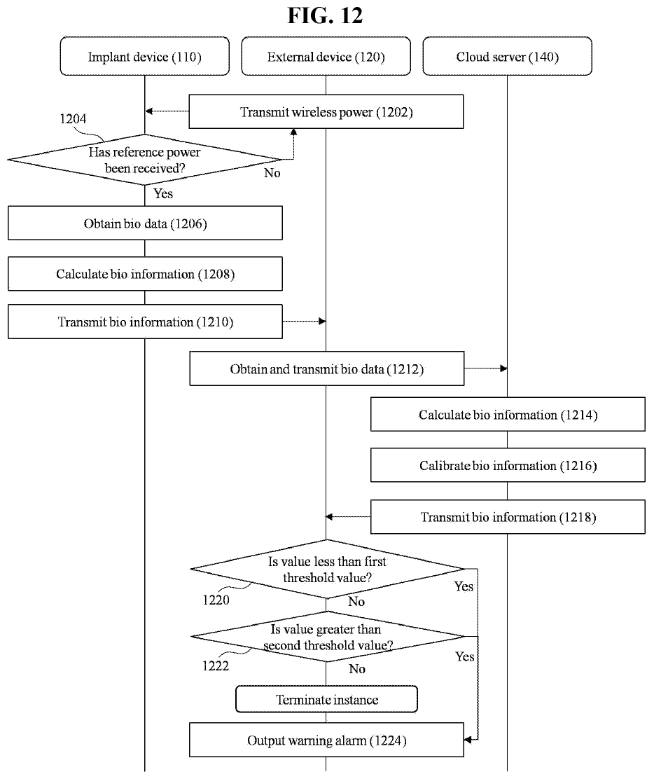

FIG. 12 is a flowchart illustrating a second example of a method of measuring bio information according to Case 2 in an embodiment of the present disclosure. FIG. 12 illustrates the implant device 110, the external device 120 and the cloud server 140.

In step 1202, the external device 120 may transmit wireless power to the implant device 110. In other words, the external device 120 may transmit the wireless power from the outside of the body to the inside of the body.

In step 1204, the implant device 110 may determine whether reference power has been received. The external device 120 transmits the wireless power from the outside of the body to the inside of the body. In this case, if the external device 120 does not accurately transmit the wireless power to a location of the implant device 110, the implant device 110 may not receive the reference power. In this case, a location of the external device 120 may be readjusted, and the wireless power may be transmitted again. To this end, if the implant device 110 does not receive the reference power, step 1202 may be performed again. If the implant device 110 receives the reference power, step 1206 may be performed.

In step 1206, the implant device 110 may obtain bio data. In this case, the implant device 110 may drive the sensing circuit by using the wireless power received from the external device 120, and may measure the bio data within the body by using the driven sensing circuit.

In step 1208, the implant device 110 may calculate bio information. In this case, the implant device 110 may calculate the bio information based on the measured bio data.

In step 1210, the implant device 110 may transmit the bio information to the external device 120.

In step 1212, the external device 120 may obtain and transmit bio data. In this case, the external device 120 may measure, outside the body, the bio data within the body through the external sensor, and may transmit the measured bio data to the cloud server 140. Furthermore, the external device 120 may further transmit, to the cloud server 130, the bio information received from the implant device 110 in step 1210.

In step 1214, the cloud server 140 may calculate bio information. For example, the cloud server 140 may receive the bio data obtained and transmitted by the external device 120 in step 1212, and may calculate the bio information based on the received bio data.

In step 1216, the cloud server 140 may calibrate (or correct) bio information. For example, the cloud server 140 may calibrate (or correct) the bio information, further received from the external device 130, based on the bio information calculated by the cloud server 140.

In step 1218, the cloud server 140 may transmit bio information to the external device 120. In this case, the transmitted bio information may be the bio information calibrated (or corrected) in step 1216. In some embodiments, the cloud server 140 may generate and/or update a history of bio information for a specific user by storing the bio information in association with the identifier of the user. In this case, the identifier of the implant device 110 and/or the identifier of the external device 120 may also be stored in the cloud server 140 in further association with the bio information. Furthermore, the cloud server 140 may transmit at least some of a generated and/or updated history to the smart device 130 or the plurality of family devices 151 to 153. Accordingly, a user or the person concerned can access the history of bio information easily and conveniently.

In step 1220, the external device 120 may determine whether a value based on the bio information (e.g., a concentration level of a target material) is less than a first threshold value. In this case, step 1222 may be performed when the corresponding value is not less than the first threshold value, and step 1226 may be performed when the corresponding value is less than the first threshold value.

In step 1222, the external device 120 may determine whether the value based on the bio information is greater than a second threshold value. In this case, a measurement instance may be terminated when the corresponding value is not greater than the second threshold value, and step 1224 may be performed when the corresponding value is greater than the second threshold value.

In step 1224, the external device 120 may output a warning alarm. The warning alarm may be output in a visual, auditory and/or tactile manner. If the value based on the bio information is the first threshold value and is smaller than a low reference level, the smart device 130 may output a warning alarm in order to give warning of a low concentration of a target material. Inversely, if the value based on the bio information is the second threshold value and is greater than a high reference level, the smart device 130 may output a warning alarm in order to give warning of a high concentration of a target material.

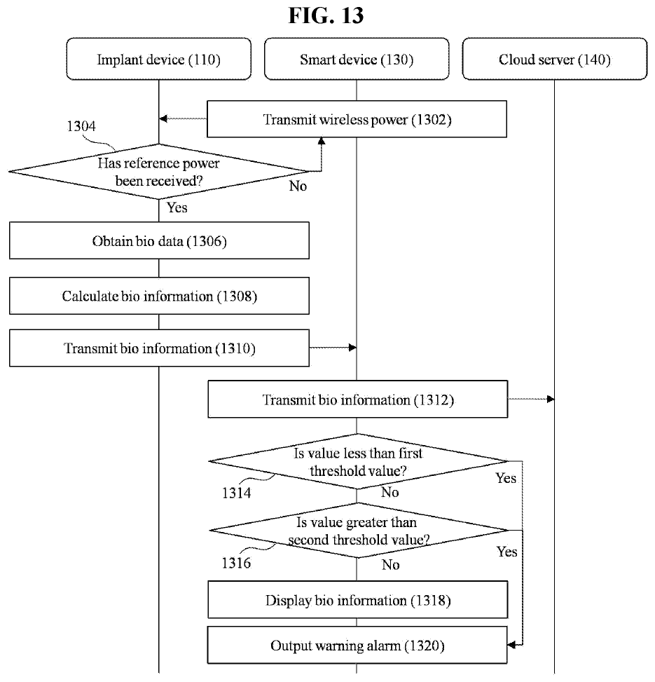

FIG. 13 is a flowchart illustrating a method of measuring bio information according to Case 3 in an embodiment of the present disclosure. FIG. 13 illustrates the implant device 110, the smart device 130 and the cloud server 140.

In step 1302, the smart device 130 may transmit wireless power to the implant device 110. In other words, the smart device 130 may transmit the wireless power from the outside of the body to the inside of the body.

In step 1304, the implant device 110 may determine whether reference power has been received. The smart device 130 transmits the wireless power from the outside of the body to the inside of the body. In this case, if the smart device 130 does not accurately transmit the wireless power to a location of the implant device 110, the implant device 110 may not receive the reference power. In this case, a location of the smart device 120 may be readjusted, and the wireless power may be transmitted again. To this end, if the implant device 110 does not receive the reference power, step 1302 may be performed again. If the implant device 110 receives the reference power, step 1306 may be performed.

In step 1306, the implant device 110 may obtain bio data. In this case, the implant device 110 may drive the sensing circuit by using the wireless power received from the smart device 130, and may measure the bio data within the body by using the driven sensing circuit.

In step 1308, the implant device 110 may calculate bio information. In this case, the implant device 110 may calculate the bio information based on the measured bio data.

In step 1310, the implant device 110 may transmit the bio information to the smart device 130.

In step 1312, the smart device 130 may transmit the bio information to the cloud server 140. The cloud server 140 may generate and/or update a history of bio information for a specific user by storing the bio information in association with an identifier of the smart device 130 and/or an identifier of the user of the smart device 130. In this case, the identifier of the implant device 110 and/or the identifier of the external device 120 may also be stored in the cloud server 140 in further association with the bio information. Furthermore, the cloud server 140 may transmit at least some of a generated and/or updated history to the smart device 130 or the plurality of family devices 151 to 153, if necessary. Accordingly, a user or the person concerned can access the history of bio information easily and conveniently.

In step 1314, the smart device 130 may determine whether a value based on the bio information (e.g., a concentration level of a target material) is less than a first threshold value. In this case, step 1316 may be performed when the corresponding value is not less than the first threshold value, and step 1320 may be performed when the corresponding value is less than the first threshold value.

In step 1316, the smart device 130 may determine whether the value based on the bio information is greater than a second threshold value. In this case, step 1318 may be performed when the corresponding value is not greater than the second threshold value, and step 1320 may be performed when the corresponding value is greater than the second threshold value.

In step 1318, the smart device 130 may display the bio information. For example, the smart device 130 may provide the bio information to a user by displaying the bio information on the display included in the smart device 130. In some embodiments, the smart device 130 may provide the bio information to a user in the form of auditory information.

In step 1320, the smart device 130 may output a warning alarm. The warning alarm may be output in a visual, auditory and/or tactile manner. If the value based on the bio information is the first threshold value and is smaller than a low reference level, the smart device 130 may output a warning alarm in order to give warning of a low concentration of a target material. Inversely, if the value based on the bio information is the second threshold value and is greater than a high reference level, the smart device 130 may output a warning alarm in order to give warning of a high concentration of a target material.

As described above, according to the embodiments of the present disclosure, the system and method for measuring bio information, which have various structures using the implant device inserted into the body, the external device and/or the smart device outside the body, can be provided.

The aforementioned system or device may be implemented as a hardware component, a software component and/or a combination of a hardware component and a software component. For example, the device and components described in the embodiments may be implemented using one or more general-purpose computers or special-purpose computers, for example, a processor, a controller, an arithmetic logic unit (ALU), a digital signal processor, a microcomputer, a field programmable gate array (FPGA), a programmable logic unit (PLU), a microprocessor or any other device capable of executing or responding to an instruction. A processing device may perform an operating system (OS) and one or more software applications executed on the OS. Furthermore, the processing device may access, store, manipulate, process and generate data in response to the execution of software. For convenience of understanding, one processing device has been illustrated as being used, but a person having ordinary knowledge in the art may understand that the processing device may include a plurality of processing components and/or a plurality of types of processing components. For example, the processing device may include a plurality of processors or one processor and one controller. Furthermore, other processing configurations, such as a parallel processor, are also possible.

Software may include a computer program, a code, an instruction or a combination of one or more of them, and may configure a processor so that it operates as desired or may instruct processors independently or collectively. Software and/or data may be embodied in any type of a machine, component, physical device, virtual equipment, or computer storage medium or device so as to be interpreted by the processor or to provide an instruction or data to the processor. The software may be distributed to computer systems connected over a network and may be stored or executed in a distributed manner. The software and data may be stored in one or more computer-readable recording media.

The method according to the embodiment may be implemented in the form of a program instruction executable by various computer means and stored in a computer-readable recording medium. The computer-readable recording medium may include a program instruction, a data file, and a data structure alone or in combination. The program instructions stored in the medium may be specially designed and constructed for the present disclosure, or may be known and available to those skilled in the field of computer software. Examples of the computer-readable storage medium include magnetic media such as a hard disk, a floppy disk and a magnetic tape, optical media such as a CD-ROM and a DVD, magneto-optical media such as a floptical disk, and hardware devices specially configured to store and execute program instructions such as a ROM, a RAM, and a flash memory. Examples of the program instructions include not only machine language code that is constructed by a compiler but also high-level language code that can be executed by a computer using an interpreter or the like.

As described above, although the embodiments have been described in connection with the limited embodiments and the drawings, those skilled in the art may modify and change the embodiments in various ways from the description. For example, proper results may be achieved although the aforementioned descriptions are performed in order different from that of the described method and/or the aforementioned components, such as the system, configuration, device, and circuit, are coupled or combined in a form different from that of the described method or replaced or substituted with other components or equivalents.

Accordingly, other implementations, other embodiments, and the equivalents of the claims fall within the scope of the claims.

1. A system for measuring bio information, comprising:
an external device configured to transmit wireless power from an outside of a body to an inside of the body;
an implant device inserted into the body and configured to drive a sensing circuit by using the wireless power transmitted by the external device, measure bio data within the body by using the driven sensing circuit, wherein measuring bio data comprises scanning electromagnetic waves in more than one electromagnetic frequency over a wide band under the skin and measuring a change in permittivity attributable to a change in blood glucose, calculate bio information based on the measured bio data, and transmit the calculated bio information to a smart device or the external device outside the body; and
the smart device configured to transmit the bio information to a cloud server or display the bio information or output a warning alarm based on the bio information, when receiving the bio information from the implant device.

2. The system of claim 1, wherein the external device comprises:
an external sensor configured to measure bio data within the body outside the body; and
a transmission module configured to transmit, to the cloud server, the bio data measured by the external sensor or bio information calculated based on the measured bio data.

3. The system of claim 2, wherein the cloud server is configured to:
receive first bio information measured by the implant device through the smart device,
receive the bio data measured by the external sensor through the external device,
calculate second bio information based on the bio data received through the external device, and
calibrate the first bio information based on the second bio information.

4. The system of claim 3, wherein the smart device is configured to:
receive the calibrated first bio information from the cloud server, and
display the calibrated first bio information or output a warning alarm based on the calibrated first bio information.

5. The system of claim 1, wherein the sensing circuit comprises both a signal source and a detector as sensing circuits having an oscillator type.

6. The system of claim 1, wherein the implant device operates under a control of Bluetooth low energy (BLE) or included in the implant device, or a micro controller unit (MCU) included in ultra low power (ULP) WiFi for communication toward the outside of the body.

7. The system of claim 1, wherein:
the implant device comprises a temperature sensor configured to measure a temperature within the body, and
the implant device calculates bio information further based on an output value of the temperature sensor.

8. The system of claim 1, wherein the implant device is configured to:
receive activity information of an object outside the body, and
calculate bio information further based on the received activity information.

9. A system for measuring bio information, comprising:
an implant device inserted into a body and configured to drive a sensing circuit by using wireless power transmitted by an external device, measure first bio data within the body by using the driven sensing circuit, wherein measuring bio data comprises scanning electromagnetic waves in more than one electromagnetic frequency over a wide band under the skin and measuring a change in permittivity attributable to a change in blood glucose, calculate first bio information based on the measured first bio data, and transmit the calculated first bio information to the external device; and
an external device configured to transmit the wireless power from an outside of the body to the implant device inserted into the body, measure, outside the body, second bio data within the body through the external sensor, receive the first bio information from the implant device, and transmit the first bio information and the second bio data to a cloud server.

10. The system of claim 9, further comprising a cloud server configured to receive the first bio information and the second bio data from the external device, calculate second bio information based on the second bio data, calibrate the first bio information based on the second bio information, and transmit the calibrated first bio information to the external device.

11. The system of claim 10, wherein the external device is configured to:
receive the calibrated first bio information transmitted by the cloud server, and
output a warning alarm based on the calibrated first bio information.

12. The system of claim 9, wherein the sensing circuit comprises both a signal source and a detector as sensing circuits having an oscillator type.

13. The system of claim 9, wherein the implant device operates under a control of Bluetooth low energy (BLE) or included in the implant device or a micro controller unit (MCU) included in ultra low power (ULP) WiFi for communication toward the outside of the body.

14. The system of claim 9, wherein:
the implant device comprises a temperature sensor configured to measure a temperature within the body, and
the implant device calculates bio information further based on an output value of the temperature sensor.

15. The system of claim 9, wherein the implant device is configured to:
receive activity information of an object outside the body, and
calculate bio information further based on the received activity information.

16. A system for measuring bio information, comprising:
an implant device inserted into a body and configured to drive a sensing circuit by using wireless power transmitted by a smart device, measure bio data within the body by using the driven sensing circuit, wherein measuring bio data comprises scanning electromagnetic waves in more than one electromagnetic frequency over a wide band under the skin and measuring a change in permittivity attributable to a change in blood glucose, calculate bio information based on the measured bio data, and transmit the calculated bio information to the smart device; and
the smart device configured to receive the bio information from the implant device and transmit the bio information to a cloud server, display the bio information or output a warning alarm based on the bio information.

17. The system of claim 16, wherein the sensing circuit comprises both a signal source and a detector as sensing circuits having an oscillator type.

18. The system of claim 16, wherein the implant device operates under a control of Bluetooth low energy (BLE) or included in the implant device or a micro controller unit (MCU) included in ultra low power (ULP) WiFi for communication toward the outside of the body.

19. The system of claim 16, wherein:
the implant device comprises a temperature sensor configured to measure a temperature within the body, and
the implant device calculates bio information further based on an output value of the temperature sensor.

20. The system of claim 16, wherein the implant device is configured to:
receive activity information of an object outside the body, and
calculate bio information further based on the received activity information.
